(19) United States
(12) Patent Application Publication
(10) Pub. No.: US 2006/0121102 A1
(43) Pub. Date: Jun. 8, 2006

(54) Transdermal systems for the delivery of estrogens and progestins

(76) Inventor: Chia-Ming Chiang, Foster City, CA (US)

Correspondence Address: REED INTELLECTUAL PROPERTY LAW GROUP, 1400 PAGE MILL ROAD, PALO ALTO, CA, 94304-1124, US

(21) Appl. No.: 11/237,284

(22) Filed: Sep. 27, 2005

Related U.S. Application Data
(60) Provisional application No. 60/613,663, filed on Sep. 27, 2004.

(51) Int. Cl. A61K 31/57; A61K 31/56; A61K 31/19; A61K 9/70

(52) U.S. Cl. 424/449; 514/171; 514/557

(57) ABSTRACT

Transdermal systems are provided for administering an estrogen and/or a progestin to a mammalian female. The systems are monolithic, having a drug reservoir that serves as the means for ensuring adhesion to the skin during drug administration. The drug reservoir includes the active agent(s), a low molecular weight organic acid as a permeation enhancer, and an additional vehicle, in an adhesive matrix. Methods for using the systems for transdermal delivery of active agents are also provided, including methods for providing hormone replacement therapy and for preventing ovulation.

### CROSS REFERENCE To RELATED APPLICATION

This application claims priority under 35 U.S.C. § 119(e)(1) to Provisional U.S. Patent Application Ser. No. 60/613,663, filed Sep. 27, 2004, which is incorporated herein by reference in its entirety.

### FIELD OF THE INVENTION

This invention relates to transdermal drug delivery, and more particularly relates to systems for administering an estrogen and/or a progestin transdermally. The invention also relates to methods of using the systems to prevent ovulation and for female hormone replacement therapy.

### BACKGROUND OF THE INVENTION

Estrogens and progestins, alone or in combination, have long been administered to female patients to prevent ovulation. Originally formulated in as oral dosage forms, these agents are now delivered transdermally or by means of am depot that is implanted beneath the skin surface. Such routes are often preferable, avoiding many of the undesirable side effects associated with oral administration.

In addition to preventing ovulation, estrogenic compounds are often administered for other purposes, including treatment of hormone deficiencies in post-menopausal women, in patients who have undergone an oophorectomy, and in patients suffering from pituitary failure. Some of these therapies are preferentially implemented with co-administration of a progestin, also referred to as a progestogen.

Transdermal administration of an estrogen such as ethinyl estradiol and gestodene is described in U.S. Pat. No. 5,376,377 to Gale et al., pertaining to a method and system for effecting contraceptive. A monoglyceride is included as a permeation enhancer, i.e., as a compound intended to facilitate transport through the skin. As is well-understood in the art, many active agents, particularly high molecular weight agents such as steroids, require administration with a permeation enhancer in order to achieve an effective blood level of the drug.

U.S. Pat. Nos. 5,876,746 and 5,972,377, both to Jona et al., describe a transdermal system and method for administering 17-deacetyl norgestimate, also referred to as norelgestromin, optionally in combination with an estrogen. The system is a patch in which the drug delivery reservoir is composed of a non-acrylate-type polymer matrix, and includes lauryl lactate as a permeation enhancer. Lauryl lactate, as will be appreciated by those in the field of transdermal delivery, is the lactic acid ester of lauryl alcohol.

U.S. Pat. No. 4,906,169 to Chien describes a transdermal estrogen/progestin dosage unit, where the estrogen is dissolved within a polymer layer, and the progestin is dissolved within a separate, adhesive layer. U.S. Pat. No. 5,762,956 to Chien describes an acrylate adhesive transdermal patch for the delivery of estrogens and progestins, and includes dimethylsulfoxide (DMSO), lauryl lactate, and ethyl lactate as permeation enhancers.

U.S. Pat. No. 5,422,119 to Casper describes a method of providing hormone replacement therapy to women by transdermally administering an estrogen with cyclically varying amounts of a progestin.

In spite of the advances in the art, there remains a need for improved transdermal delivery systems for the delivery of steroids such as estrogens and progestins, in which drug administration is efficient, i.e., exhibiting a high rate of transport, or “flux,” through the skin, and unwanted side effects are minimized. An ideal transdermal drug delivery system for the administration of steroids would exclude any potentially toxic vehicles or enhancers (such as DMSO) and be readily manufacturable using straightforward means, for instance avoiding the inclusion of multiple layers. The present invention provides such a system.

### SUMMARY OF THE INVENTION

In one aspect of the invention, a transdermal system is provided for the delivery of an estrogen and/or a progestin, wherein the system is “monolithic” insofar as a single drug reservoir layer is present which also serves as the means for adhering the system to a body surface. The system is thus composed of a backing layer and a drug reservoir layer that contains an effective amount of the active agent(s), a low molecular weight organic acid as a permeation enhancer, and an additional vehicle (which may be an additional enhancer) in an adhesive matrix selected from acrylate adhesives, silicone adhesives, and polyisobutylene adhesives.

In other aspects of the invention, a method is provided for using the aforementioned transdermal system to effect drug delivery, for instance in the prevention of ovulation or in providing hormone replacement therapy.

### BRIEF DESCRIPTION OF THE DRAWINGS

FIG. 1 depicts the in vitro skin flux of norelgestromin for several formulations of the invention.

FIG. 2 and FIG. 3 depict the in vitro skin flux of norelgestromin and ethinyl estradiol for a formulation of the invention, as compared to the Ortho Evra® norelgestromin/ethinyl estradiol transdermal system (Ortho-McNeil Pharmaceutical, Inc.).

FIG. 4 depicts the in vitro skin flux of norelgestromin and ethinyl estradiol for another formulation of the invention, as compared to the Ortho Evra® transdermal system.

FIG. 5 and FIG. 6 depict the in vitro skin flux of ethinyl estradiol and norelgestromin, respectively, for another formulation of the invention, as compared to the Ortho Evra® transdermal system.

FIG. 7 and FIG. 8 depict the in vitro skin flux of ethinyl estradiol and norelgestromin, respectively, for another formulation of the invention, as compared to the Ortho Evra® transdermal system.

FIGS. 9A-9B and FIGS. 10A-10B depict the in vitro skin flux of norelgestromin and ethinyl estradiol for two other formulations of the invention, as compared to the Ortho Evra® transdermal system.

### DETAILED DESCRIPTION OF THE INVENTION

Terminology:

Before describing detailed embodiments of the invention, it will be useful to set forth definitions that are used in describing the invention. The definitions set forth apply only to the terms as they are used in this patent and may not be applicable to the same terms as used elsewhere, for example in scientific literature or other patents or applications including other applications by these inventors or assigned to common owners. The following description of the preferred embodiments and examples are provided by way of explanation and illustration. As such, they are not to be viewed as limiting the scope of the invention as defined by the claims. Additionally, when examples are given, they are intended to be exemplary only and not to be restrictive. For example, when an example is said to “include” a specific feature, that is intended to imply that it may have that feature but not that such examples are limited to those that include that feature.

As used in this specification and the appended claims, the singular forms “a”, “an” and “the” include plural referents unless the context clearly dictates otherwise. Thus, for example, reference to “an estrogen” includes a single estrogenic compound as well as a combination or mixture of two or more different estrogenic compounds, reference to “a pharmaceutically acceptable vehicle” includes a mixture of two or more such vehicles as well as a single vehicle, reference to “a permeation enhancer” includes mixtures of two or more such enhancers as well as a single enhancer, and the like.

In describing and claiming the present invention, the following terminology will be used in accordance with the definitions set out below.

The term “active agent” refers to a chemical material or compound suitable for transdermal administration and that induces a desired effect. The terms include agents that are therapeutically effective and prophylactically effective. Also included are pharmaceutically acceptable, pharmacologically active derivatives of those active agents specifically mentioned herein, including, but not limited to, salts, esters, amides, prodrugs, active metabolites, inclusion complexes, analogs, and the like, which also induce the desired effect. The terms “active agent,” “drug” and “therapeutic agent” are used interchangeably herein.

By “transdermal” delivery is meant administration of an active agent to a body surface of an individual so that the agent passes through the body surface, e.g., skin, and into the individual's blood stream. The term “transdermal” is intended to include transmucosal administration, i.e., administration of a drug to the mucosal (e.g., sublingual, buccal, vaginal, rectal) surface of an individual so that the agent passes through the mucosal tissue and into the individual's blood stream. The term “body surface” is used to refer to skin or mucosal tissue, including the interior surface of body cavities that have a mucosal lining. The term “skin” should be interpreted as including “mucosal tissue” and vice versa. Preferred body surfaces are intact areas of skin.

The term “effective amount” is intended to mean the amount of an active agent that is nontoxic but sufficient to provide the desired effect, and includes both “therapeutically effective” and “prophylactically effective” amounts. Similarly, an “ovulation-inhibiting amount” is intended to mean the amount of an active agent that is nontoxic but sufficient to inhibit ovulation in a female patient. The amount that is “effective” will vary from subject to subject, depending on the age and general condition of the individual, the particular active agent or agents, and the like. Thus, it is not always possible to specify an exact effective amount. However, an appropriate effective amount in any individual case may be determined by one of ordinary skill in the art using routine experimentation. Furthermore, the exact effective amount of an active agent incorporated into the adhesive of the invention is not critical, so long as the concentration is within a range sufficient to permit ready application of the formulation so as to deliver an amount of the active agent that is within a therapeutically effective range.

Drug Delivery Systems:

The transdermal system of the invention is a monolithic system comprising an outwardly facing backing layer, and, laminated thereto, an inwardly facing, body surface contacting, drug reservoir layer. During storage and prior to use, the system also includes a readily removable release liner protecting the skin-contacting surface of the drug reservoir layer. The systems are adapted to be in diffusional communication with a body surface so as to administer the desired amount of active agent across the body surface.

The transdermal system is suitable for implementation in any method involving the administration of steroids, particularly the administration of sex steroids such as estrogens, progestins, and androgens. For instance, the system finds utility in the prevention of ovulation and for providing hormone replacement therapy (HRT), and is implemented to deliver either an ovulation-inhibiting amount or a effective HRT-providing amount of the active agent across a body surface.

In one embodiment, the system comprises a backing layer and an adhesive layer underlying and laminated to the backing layer, wherein the adhesive layer contains the active agent(s), a low molecular weight organic acid as a permeation enhancer, and an additional pharmaceutically acceptable vehicle which may be an additional permeation enhancer. The adhesive layer of the system is an adhesive matrix that functions as the drug reservoir and the skin contact adhesive means, and comprises about 1 wt. % to about 20 wt. %, preferably about 2 wt. % to about 15 wt. % of the active agent(s).

Preferred active agents are sex steroids, particularly estrogens, progestins, and androgens, with estrogens and progestins particularly preferred.

There are numerous estrogen compounds that can be administered using the transdermally systems of the invention. These include naturally occurring estrogens such as 17-β-estradiol and estrone, as well as synthetic and semi-synthetic derivatives of natural estrogens, e.g., of 17-β-estradiol, providing that those derivatives are biocompatible and can be delivered transdermally in therapeutically or prophylactically effective amounts. In addition, non-steroidal compounds having estrogenic activity can also be delivered using the present systems. These include, by way of illustration, diethylstilbestrol, dienestrol, clomifen, chlorotrianisene, and cyclofenil.

Suitable derivatives of estradiol include mono-esters (either 3- or 17β-esters) and di-esters, such as, for example: estradiol-17β-enanthate; estradiol-3,17-diacetate; estradiol-3-acetate; estradiol-17-acetate; estradiol-3,17-divalerate; estradiol-3-valerate; estradiol-17β-valerate; 3-mono, 17-mono and 3,17-dipivilate esters; 3-mono, 17-mono and 3,17-dipropionate esters; 3-mono, 17-mono and 3,17-di-cyclopentyl-propionate esters; the corresponding cypionate, heptanoate, undecanoate, and benzoate esters, e.g., estradiol-17β-cypionate, estradiol-17β-undecanoate, and estradiol-3-benzoate; 17-alkylated estrogens, such as ethinyl estradiol, ethinyl estradiol-3-isopropylsulphonate, quinestrol, mestranol and methyl estradiol; estradiol-16,17-hemisuccinate; and (19-norpregna-1,3,5 (10)-trien-20-yne-3,17-diol). Combinations of these estrogens may also be delivered using the present systems. A preferred estrogen is ethinyl estradiol (EE).

There are numerous progestin compounds that can be delivered using the present systems, including, by way of example, desogestrel, dihydroprogesterone, ethynodiol acetate, ethynodiol diacetate, gestodene, gestogen, 17-hydrogesterone, hydroxyprogesterone caproate, 3-keto-desogestrel, levonorgestrel, medroxyprogesterone acetate, medroxyprogesterone diacetate, megestrol, megestrol acetate, normegesterol, norelgestromin, norethindrone, norethindrone acetate, norethynodrel, norgestimate, norgestrel, and progesterone, and combinations thereof. As noted, derivatives of the aforementioned progestins may also be used. These include pharmacologically acceptable derivatives such as ethers, esters, amides, acetals, salts and the like. A preferred progestin is norelgestromin (NGMN), which is also referred to as 18,19-dinorpregn-4-en-20-yn-3-one,13-ethyl-17-hydroxy-3-oxime or 17-deacetyl norgestimate.

Androgenic steroids may, in some cases, be included in the transdermal drug delivery systems of the invention, particularly in hormone replacement therapy. Preferred androgens are testosterone, testosterone esters (e.g., the enanthate and propionate esters), dehydroepiandrosterone (DHEA; also termed “prasterone”), sodium dehydroepiandrosterone sulfate, and 4-dihydrotestosterone (DHT). The amount of androgenic agent in the transdermal systems, if any is present, will generally be about half that of the progestin, by weight, although the exact amount will obviously depend on the particular androgenic agent and its potency.

The drug reservoir also includes a low molecular weight organic acid (preferred molecular weight is in the range of about 60 to about 200), which serves as a permeation enhancer. Examples of such acids include lactic acid, citric acid, glycolic acid, malic acid, tartaric acid, fumaric acid, mandelic acid, pyruvic acid, itaconic acid, malonic acid, succinic acid, oxalic acid, butyric acid, octanoic acid, alpha-hydroxyethanoic acid, alpha-hydroxyoctanoic acid, caprylic acid, and alpha-hydroxy caprylic acid. Particularly preferred acids are alpha-hydroxy acids, i.e., acids in which the carboxylic acid group COOH is bound to a carbon atom also substituted with a hydroxyl group. Preferred alpha-hydroxy acids include, without limitation, lactic acid, citric acid, glycolic acid, malic acid, and tartaric acid, with lactic acid particularly preferred. Chiral acids may be present in the form of a racemate (e.g., mesotartaric acid) or in enantiomerically pure form (e.g., levotartaric or dextrotartaric acid).

The amount of the organic acid in the drug reservoir is an effective permeation enhancing amount, meaning that the amount is sufficient to provide the desired drug delivery profile. A higher loading will provide a higher drug flux at an early time period, with a decrease in flux over time. A lower loading provides a steady state flux. It has been found that a low molecular weight organic acid such as lactic acid has proven particularly effective in enhancing the transdermal flux of norelgestromin, as compared with other enhancers such as lauryl lactate.

Generally, the permeation enhancer should be incorporated so as to represent about 0.5 wt. % to about 15 wt. % of the drug reservoir, preferably about 2 wt. % to about 10 wt. %, and optimally about 1 wt. % to about 5 wt. %.

The drug reservoir layer additionally comprises from about 2 wt. % to about 40 wt %, preferably from about 2 wt. % to about 30 wt % and more preferably from about 2 wt. % to about 20 wt % of at least one additional pharmaceutically acceptable vehicle, which may be an additional permeation enhancer. Vehicles may also be selected to facilitate solubilization of the active agent or other reservoir components, promote homogeneous admixture of reservoir components, and/or facilitate manufacture.

Exemplary pharmaceutically acceptable vehicles include, by way of illustration and not limitation, C₁₋₁₈ branched, linear, cyclic, saturated and unsaturated monohydric alcohols; polyols (including diols) and esters thereof such as propylene glycol (PG); N-methylpyrrolidone; C₁₋₄ alkanol esters of lactic acid such as ethyl lactate; and combinations thereof.

Examples of unbranched monohydric alcohols include methanol, ethanol, denatured ethanol, propanol, butanol, pentanol, hexanol, heptanol, octanol, nonanol, decanol, undecanol, dodecanol (i.e., lauryl alcohol), tridecanol, tetradecanol (i.e., myristyl alcohol), pentadecanol and hexadecanol (i.e., palmityl alcohol) are preferred. Other preferred monohydric alcohols include isopropyl alcohol, isobutyl alcohol, s-butyl alcohol, t-butyl alcohol, cyclohexanol, phenol, benzyl alcohol, and so forth. The monohydric alcohol can be optionally substituted with 1 to 4 substituents such as halo, lower alkoxy, thiol, and so on.

Particularly well suited vehicles include, by way of illustration and not limitation, propylene glycol, N-methylpyrrolidone (NMP), and ethyl lactate.

Additional vehicles which can serve as co-permeation enhancers along with the organic acid are selected to further enhance the flux of one or more of the active agents in the drug reservoir. Selection of suitable permeation enhancers will depend upon the particular active agent or agents being delivered, as well as the enhancer's compatibility with the other components of the adhesive.

Exemplary permeation enhancers include, by way of illustration and not limitation, alcohols such as ethanol, propanol, octanol, decanol or n-decyl alcohol, benzyl alcohol, and the like; alkanones; amides and other nitrogenous compounds such as urea, dimethylacetamide, dimethylformamide, 2-pyrrolidone, 1-methyl-2-pyrrolidone, ethanolamine, diethanolamine and triethanolamine; 1-substituted azacycloheptan-2-ones, particularly 1-n-dodecylcyclazacycloheptan-2-one; bile salts; cholesterol; cyclodextrins and substituted cyclodextrins such as dimethyl-β-cyclodextrin, trimethyl-β-cyclodextrin and hydroxypropyl-β-cyclodextrin; ethers such as diethylene glycol monoethyl ether (available commercially as Transcutol®) and diethylene glycol monomethyl ether; fatty acids, both saturated and unsaturated, such as lauric acid, oleic acid and valeric acid; fatty acid esters, both saturated and unsaturated, such as isopropyl myristate, isopropyl palmitate, methylpropionate, and ethyl oleate; fatty alcohol esters, both saturated and unsaturated, such as the fatty C₈₋₂₀ alcohol esters of lactic acid (e.g., lauryl lactate or propanoic acid 2-hrdroxy-dodecyl ester); glycerides such as labrafil and triacetin, and monoglycerides such as glycerol monooleate, glycerol monolinoleate and glycerol monolaurate, as described in U.S. Pat. No. 5,376,377 to Gale et al.; organic acids, particularly salicylic acid and salicylates, citric acid and succinic acid; methyl nicotinate; pentadecalactone; polyols and esters thereof such as propylene glycol, ethylene glycol, glycerol, butanediol, polyethylene glycol, and polyethylene glycol monolaurate; phospholipids such as phosphatidyl choline, phosphatidyl ethanolamine, dioleoylphosphatidyl choline, dioleoylphosphatidyl glycerol and dioleoylphoshatidyl ethanolamine; sulfoxides such as dimethylsulfoxide (DMSO) and decylmethylsulfoxide (C₁₀MSO); surfactants such as sodium laurate, sodium lauryl sulfate, cetyltrimethylammonium bromide, benzalkonium chloride, Poloxamer® (231, 182, 184), poly(oxyethylene) sorbitans such as Tween® (20, 40, 60, 80) and lecithin; terpenes; and combinations thereof. Particularly well suited enhancers include, by way of illustration and not limitation, fatty acid esters; and glycerides such as lauryl lactate, labrafil and triacetin.

The adhesive material providing the adhesive matrix of the drug reservoir can be an acrylate adhesive, a silicone adhesive, or a polyisobutylene (PIB) adhesive, all of which are well known in the art.

Acrylate adhesives are typically made by copolymerizing at least one acrylate or methacrylate monomer with at least one modifying monomer and at least one functional group-containing monomer. Exemplary acrylate and methacrylate monomers include 2-ethylhexyl acrylate, butyl acrylate, and isooctyl acrylate. Exemplary modifying monomers include vinyl acetate, ethyl acrylate, methacrylate, and methyl methacrylate. Exemplary functional groups include carboxyl and hydroxy groups such as those present on acrylic acid, methacrylic acid and hydroxy-containing monomers such as hydroxyethyl acrylate. Particularly suitable acrylate-based adhesives comprise polyacrylate adhesive copolymers such as those comprising a 2-ethylhexyl acrylate monomer. Suitable acrylate-based adhesives include those commercially available from the National Starch and Chemical Company under the trademark Duro-Tak®. These include, for example: Duro-Tak 87-900A, an acrylic non-curing pressure sensitive adhesive supplied in an organic solvent (ethyl acetate); Duro-Tak 87-2287 and 87-4287, acrylate-vinyl acetate non-curing pressure sensitive adhesives supplied in an organic solvent solution; and Duro-Tak 87-2516, an acrylate-vinyl acetate self-curing pressure sensitive adhesive supplied in an organic solvent solution.

Silicone adhesives are typically made from silicone polymers that are cross-linkable at room temperature. The silicone polymers can have a block or graft structure or a combination of both. Thus, the silicone polymers can have one block of dimethylsiloxane units, with another block made up of different repeating (e.g., methylvinylsiloxane, diphenylsiloxane, diisopropyl siloxane units or other siloxane or silane units). Numerous examples of suitable silicone materials are described in U.S. Pat. No. 4,906,169 to Chien and U.S. Pat. No. 5,232,702 to Pfister et al. Exemplary crosslinking agents and catalysts include: tetrapropoxy silane [Si(OCH₂CH₂CH₃)₄] for silicone polymers having free hydroxy groups such as terminal hydroxy groups; dimethyl-silicone polymers using a catalyst such as a platinum catalyst for silicone polymers having vinyl groups; peroxide catalysts for crosslinking silicone copolymers having dimethyl and methylvinyl siloxane units. Particularly suitable silicone-based adhesives comprise polydimethyl siloxanes or polydimethyldiphenyl siloxanes.

Suitable silicone adhesives include those commercially available from Dow Corning such as Silastic 382, Q7-4635, Q7-4650, Q7-4665, Q7-4735, Q7-4750, Q7-4765 and MDX-4-4210.

Polyisobutylene-based adhesives are typically made from mixtures of high molecular weight polyisobutylenes (700,000-2,000,000 Da) and low molecular weight polyisobutylenes (35,000-60,000 Da). The weight ratio of high to low molecular weight polyisobutylene in the adhesive will typically be 1:1 to 1:10. Polyisobutylene-based adhesives will usually include a tackifier such as polybutene oil and an aliphatic resins such as the ESCOREZ® resins (Exxon Chemical).

Suitable polyisobutylene adhesives include those commercially available from ExxonMobil Chemical under the trademark VISTANEX™ or from BASF under trademark Oppanol™.

The drug reservoir may also include one or more optional components, e.g., hydrophilic, water-absorbing materials that improve the wear properties of the system. Such materials are also referred to as matrix modifiers, and examples include hydrophilic polymers having repeating units derived from an N-vinyl lactam monomer, a carboxy vinyl monomer, a vinyl ester monomer, an ester of a carboxy vinyl monomer, a vinyl amide monomer, and/or a hydroxy vinyl monomer, such as, by way of example, poly(N-vinyl lactams), poly(N-vinyl acrylamides), poly(N-alkylacrylamides), substituted and unsubstituted acrylic and methacrylic acid polymers (e.g., polyacrylic acids and polymethacrylic acids), polyvinyl alcohol (PVA), polyvinylamine, copolymers thereof and copolymers with other types of hydrophilic monomers (e.g. vinyl acetate); cross-linked polymers such as cross-linked polyvinyl pyrrolidones, cross-linked carboxy methyl cellulose, and so forth; starch and starch derivatives; cellulosics such as carboxymethylcellulose and hydroxypropylmethylcellulose, as well as cellulose esters such as cellulose acetate, cellulose acetate propionate, cellulose acetate butyrate, cellulose propionate, cellulose butyrate, cellulose propionate butyrate, cellulose diacetate, and cellulose triacetate; alginic acid; chitosan; gelatin and so forth.

Preferred matrix modifiers include: homopolymers or copolymers of N-vinyl lactam monomer units, with N-vinyl lactam monomer units representing the majority of the total monomeric units of a poly(N-vinyl lactams) copolymer; and cross-linked polymers such as cross-linked polyvinyl pyrrolidones. Preferred poly(N-vinyl lactams) for use in conjunction with the invention are prepared by polymerization of one or more of the following N-vinyl lactam monomers: N-vinyl-2-pyrrolidone; N-vinyl-2-valerolactam; and N-vinyl-2-caprolactam. Particularly preferred matrix modifiers include polyvinyl pyrrolidone and cross-linked polyvinyl pyrrolidone.

Matrix modifiers can be present in an amount of about 0-20 wt % of the drug reservoir layer, preferably 5-10 wt %.

Other optional components which may be in the drug reservoir include conventional additives such as absorbent or inert fillers, excipients, preservatives, pH regulators, plasticizers, softeners, thickeners, stabilizers, tackifiers or adhesive agents, and antioxidants.

Absorbent fillers may be advantageously incorporated to control the degree of hydration of the adhesive layer. Such fillers can include microcrystalline cellulose, talc, clay, lactose, guar gum, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, and woven, non-woven paper, and cotton materials. Other suitable fillers are inert, i.e., substantially non-adsorbent, and include, for example, polyethylenes, polypropylenes, polyurethane polyether amide copolymers, polyesters and polyester copolymers, nylon, and rayon. One preferred filler is colloidal silica, e.g., Cab-O-Sil® (available from Cabot Corporation, Boston Mass.).

Preservatives include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

Compounds useful as pH regulators include, but are not limited to, glycerol buffers, citrate buffers, borate buffers, phosphate buffers, and citric acid-phosphate buffers, which may be included so as to ensure that the pH of the composition is compatible with that of an individual's body surface.

Suitable plasticizers and softeners include, by way of illustration and not limitation, alkyl and aryl phosphates such as tributyl phosphate, trioctyl phosphate, tricresyl phosphate, and triphenyl phosphate; alkyl citrate and citrate esters such as trimethyl citrate, triethyl citrate and acetyl triethyl citrate, tributyl citrate and acetyl tributyl citrate, acetyl triethyl citrate, and trihexyl citrate; alkyl glycerolates; alkyl glycolates; dialkyl adipates such as dioctyl adipate (also referred to as bis(2-ethylhexyl)adipate), diethyl adipate, di(2-methylethyl)adipate, and dihexyl adipate; dialkyl phthalates, dicycloalkyl phthalates, diaryl phthalates and mixed alkyl-aryl phthalates, including phthalic acid esters, as represented by dimethyl phthalate, diethyl phthalate, dipropyl phthalate, dibutyl phthalate, di(2-ethylhexyl)-phthalate, di-isopropyl phthalate, diamyl phthalate and dicapryl phthalate; dialkyl sebacates such as diethyl sebacate, dipropyl sebacate, dibutyl sebacate and dinonyl sebacate; dialkyl succinates such as diethyl succinate and dibutyl succinate; dialkyl tartrates such as diethyl tartrate and dibutyl tartrate; glycol esters and glycerol esters such as glycerol diacetate, glycerol triacetate (triacetin), glycerol monolactate diacetate, methyl phthalyl ethyl glycolate, butyl phthalyl butyl glycolate, ethylene glycol diacetate, ethylene glycol dibutyrate, triethylene glycol diacetate, triethylene glycol dibutyrate and triethylene glycol dipropionate; hydrophilic surfactants, preferably hydrophilic non-ionic surfactants such as, for example, partial fatty acid esters of sugars, polyethylene glycol fatty acid esters, polyethylene glycol fatty alcohol ethers, and polyethylene glycol sorbitan-fatty acid esters, as well as non-ionic surfactants such as ethylcellosolve; lower alcohols from ethyl to octyl; lower diols such as 1,2- and 1,3-propylene glycol; low molecular weight poly(alkylene oxides) such as polypropylene glycol and polyethylene glycol; polyhydric alcohols such as glycerol; sorbitol; tartaric acid esters such as dibutyl tartrate; and mixtures thereof.

Suitable thickeners are naturally occurring compounds or derivatives thereof, and include, by way of example, collagen, galactomannans, starches, starch derivatives and hydrolysates, cellulose derivatives such as methyl cellulose, hydroxypropylcellulose, hydroxyethyl cellulose, and hydroxypropyl methyl cellulose, colloidal silicic acids, and sugars such as lactose, saccharose, fructose and glucose. Synthetic thickeners such as polyvinyl alcohol, vinylpyrrolidone-vinylacetate-copolymers, polyethylene glycols, and polypropylene glycols, may also be used.

Suitable stabilizers include, parabens such as methyl paraben and propyl paraben.

Suitable tackifiers or adhesive agents can also be included to improve the adhesive and tack properties, and may be solid or liquid. Exemplary materials include tacky rubbers such as polyisobutylene, polybutadiene, butyl rubber, polystyrene-isoprene copolymers, polystyrene-butadiene copolymers, and neoprene (polychloroprene). Preferred adhesive agents include low molecular weight polyisobutylene and butyl rubber. Other examples of suitable tackifiers herein are those that are conventionally used with pressure sensitive adhesives, e.g., rosins, rosin esters (for example Sylvagum® RE 85K (formerly Zonester® 85K Resin) available from Arizona Chemical), polyterpenes, and hydrogenated aromatic resins in which a very substantial portion, if not all, of the benzene rings are converted to cyclohexane rings (for example, the Regalrez family of resins manufactured by Hercules, such as Regalrez 1018, 1033, 1065, 1078 and 1126, and Regalite R-100, the Arkon family of resins from Arakawa Chemical, such as Arkon P-85, P-100, P-115 and P-125) and hydrogenated polycyclic resins (typically dicyclopentadiene resins, such as Escorez 5300, 5320, 5340 and 5380 manufactured by Exxon Chemical Co.).

Antioxidants can be included in the adhesive layer to protect against light-induced oxidation, chemically induced-oxidation, and thermally-induced oxidative degradation during processing and/or storage. Oxidative degradation, as will be appreciated by those in the art, involves generation of peroxy radicals, which in turn react with organic materials to form hydroperoxides. Primary antioxidants are peroxy free radical scavengers, while secondary antioxidants induce decomposition of hydroperoxides, and thus protect a material from degradation by hydroperoxides. Most primary antioxidants are sterically hindered phenols, and preferred such compounds for use herein are tetrakis [methylene (3,5-di-tert-butyl-4-hydroxyhydrocinnamate)] methane (e.g., Irganox® 1010 available from Ciba-Geigy Corp., Hawthorne, N.Y.) and 1,3,5-trimethyl-2,4,6-tris [3,5-di-t-butyl-4-hydroxy-benzyl] benzene (e.g., Ethanox® 330 available from Ethyl Corp.). A particularly preferred secondary antioxidant that may replace or supplement a primary antioxidant is tris(2,4-di-tert-butylphenyl)phosphite (e.g., Irgafos® 168 available from Ciba-Geigy Corp.). Multi-functional antioxidants are also useful, and serve as both a primary and a secondary antioxidant. Irganox® 1520 D, manufactured by Ciba-Geigy is one example of a multifunctional antioxidant. Vitamin E antioxidants, such as that sold by Ciba-Geigy as Irganox® E17, are also useful in the present compositions. Other suitable antioxidants include, without limitation, ascorbic acid, ascorbic palmitate, tocopherol acetate, propyl gallate, butylhydroxyanisole (BHA), butylated hydroxytoluene (BHT), bis(1,2,2,6,6-pentamethyl-4-piperidinyl)-(3,5-di-tert-butyl-4-hydroxybenzyl)butylpropanedioate, (available as Tinuvin® 144 from Ciba-Geigy Corp.) and a combination of octadecyl 3,5-di-tert-butyl-4-hydroxyhydrocinnamate (also known as octadecyl 3-(3′,5′-di-tert-butyl-4′-hydroxyphenyl)propionate) (Naugard® 76 available from Uniroyal Chemical Co., Middlebury, Conn.) and bis(1,2,2,6,6-pentamethyl-4-piperidinylsebacate) (Tinuvin® 765 available from Ciba-Geigy Corp.).

The backing layer of the drug delivery system of the invention functions as the primary structural element and provides the system with flexibility. The material used for the backing layer should be inert and substantially impermeable to the active agent(s), vehicle, permeation enhancer, and other components of the adhesive layer. Also, the material used for the backing layer should permit the system to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device.

The backing layer is preferably in the range of about 15μ to about 250μ in thickness, and may, if desired, be pigmented, metallized, or provided with a matte finish suitable for writing.

The backing layer is preferably non-occlusive (or “breathable”), i.e., is preferably permeable to moisture. Typically the systems are intended to be worn for several days and use of a non-occlusive backing permits passage of both sweat and air to minimize hydration of the body surface. However, occlusive backing materials can also be used. The backing layer faces outward, away from the body surface. Thus, the outer surface is preferably non-tacky, while the inner surface may be tacky to facilitate adherence to the adhesive drug reservoir.

Examples of materials useful as the backing layer include, by way of illustration and not limitation, ethylene vinyl acetate, polyesters such as poly(ethylene phthalate) and polyethylene terephthalate, polyether amides, polyethylenes, polyolefins such as ethylene-vinyl acetate copolymers, polypropylenes, polyurethanes, polyvinylchlorides, polyvinylidene chlorides, and combinations thereof.

Other suitable materials are non-polymeric, e.g., metal foils, metal foil laminates foils (e.g., laminates of polymer films with metallic foils such as aluminum foil), waxes (e.g., microcrystalline or paraffin waxes), and wax/foam laminates. The backing layer can also be an open-cell foam such as a polyurethane, polystyrene or polyethylene foam.

There are many commercially available materials that are well suited for use as backing layers for the systems of the invention. Particularly preferred materials are commercially available materials such as the polyester-based laminates sold under the name 3M™ Scotchpak™ 9723, 9732 or 9733, by 3M Drug Delivery Systems.

The release liner is a disposable element that serves to cover the otherwise exposed surface of the drug reservoir and thus protects the system during storage and prior to application to the body surface. The release liner is preferably formed from a material impermeable to the drug, enhancer or other components of the matrix, and that is easily stripped from the matrix. Release liners are typically treated with silicone or fluorocarbons, and are commonly made from polyesters and polyethylene terephthalate.

There are many commercially available materials that are well suited for use as release liners such as the Scotchpak® materials sold by the 3M Company and the Bio-Release® materials sold by Dow Corning. Of particular interest are the siliconized release materials sold by Loparex Inc.

Methods of Manufacture:

In general, the active agents and other matrix components are mixed, cast onto the backing layer and dried to form a thin layer. The release liner is then laminated onto the adhesive reservoir.

The drug polymer matrix can be formed by simply dissolving or otherwise finely dispersing the drug with vehicles/enhancers in a polymer solution to yield a solution or slurry, casting the slurry or solution and then evaporating the volatile solvents to give a solid drug polymer matrix with drug incorporated therein.

Conventional polymer solution-handling equipment such as mixers, mills or the like, are used to make a homogeneous solution or suspension of the drug polymer mixture. The process can be completed in from a few seconds to a few hours, depending upon mixing conditions.

The casting can be carried out using manual casting machines or doctor blades or the like or can be carried out with commercial film casting equipment for large scale production.

Solvent removal is carried out using heat, air flow and/or a vacuum. Temperatures are preferably held below temperatures at which significant degradation of drug occurs and the vehicles enhancers will not evaporated. Typically, suitable temperatures range from room temperature (approximately 20-25° C.) to about 100° C., although higher temperatures can be used. Solvent removal should be completed until no substantial solvent remains. The thickness of the resultant drug polymer matrix can vary from 10 micrometers to about 250 micrometers. Preferred thicknesses are from 15 to 100 micrometers.

An example of solution casting involves admixing the components of the matrix in a suitable solvent, e.g., a volatile solvent such as ethyl acetate, or lower alkanols (e.g., ethanol, isopropyl alcohol, etc.), at a concentration typically in the range of about 35-60% w/v. The solution is cast onto a suitable substrate such as the backing layer or release liner. Both admixture and casting are carried out at ambient temperature. The substrate coated with the film is then baked at a temperature of about 90° C., for time of about two hours.

The systems will typically be individually wrapped as a unit dosage form, with several unit dosage forms packed in the same box, with appropriate written instructions for use.

Methods of Use:

The transdermal system of the invention is useful for preventing ovulation and for providing hormone replacement therapy in a female. Ovulation-inhibiting dosages of estrogen and progestin, as well as the therapeutically effective amount of estrogen and progestin needed to provide satisfactory hormone replacement therapy, will vary depending upon the particular active agent being administered. While the determination of such amounts are well within the knowledge of those skilled in the art, the following examples will provide some guidance for the preferred active agents.

| ACTIVE AGENT | EXEMPLARY DOSAGE |
|---|---|
| ethinyl estradiol | about 10-35 μg/day |
| 17-β-estradiol | about 25-150 μg/day, preferably about 25-50 |
| | μg/day |
| norelgestromin | about 150-350 μg/day, preferably about 175-300 |
| | μg/day |
| norgestimate | about 100-1500 μg/day, preferably about 125-250 |
| | μg/day |
| norethindrone | about 1000 μg/day |
| norethindrone | about 25-1000 μg/day, preferably about 75-500 |
| acetate | μg/day |
| 3-keto-desogestrel | about 5-150 μg/day, preferably about 25-150 |
| | μg/day |

The transdermal system dosage unit is typically formulated so as to provide at least minimum daily doses of the estrogen and progestin for multiple days. Therefore, whether intended for use to prevent ovulation or for providing hormone replacement therapy, the systems are intended to deliver the active agent(s) to the body surface continuously for an extended time period, which will typically be from 1 to 7 days, and preferably for about 7 days.

When the system is used to prevent ovulation, the system will typically be placed on the body surface on the fifth day of the female's menstrual cycle, and replaced as needed until twenty-one days of wearing have elapsed. For example, for a 7-day system, three systems will be used to deliver the active agents for the full 21-day period. If desired a placebo system may be worn thereafter until the fifth day of the succeeding menstrual cycle. This regimen is then repeated for each menstrual cycle for as long as ovulation prevention is desired.

Typically, each new system is applied to a different site on the body surface. Suitable application sites include, below the waistline such as the buttock, hip or abdominal area. The body surface is preferably cleaned and dried before application of the system.

### EXAMPLES

The practice of the present invention will employ, unless otherwise indicated, conventional techniques of synthetic organic and medicinal chemistry, pharmaceutical formulation, and the like, which are within the skill of the art. Such techniques are explained fully in the literature. Such techniques are explained fully in the literature. See, for example, Kirk-Othmer's Encyclopedia of Chemical Technology; and House's Modern Synthetic Reactions. Preparation of various types of pharmaceutical formulations are described, for example, in Remington: The Science and Practice of Pharmacy, 20th edition (Lippincott Williams & Wilkins, 2000) and Ansel et al., Pharmaceutical Dosage Forms and Drug Delivery Systems, 6th Ed. (Media, Pa.: Williams & Wilkins, 1995).

In the following examples, efforts have been made to insure accuracy with respect to numbers used (e.g., amounts, temperature, etc.) but some experimental error and deviation should be accounted for. Unless indicated otherwise, temperature is in degrees C. and pressure is at or near atmospheric. All solvents were purchased as HPLC grade, and all reactions were routinely conducted under an inert atmosphere of argon unless otherwise indicated. All reagents were obtained commercially unless otherwise indicated.

### Example 1

The in vitro flux of norelgestromin (NGMN) from the following formulations (amounts shown as wt %) was evaluated. The adhesive was an acrylate adhesive, commercially available from the National Starch and Chemical Company under the name Duro-Tak® 87-4287. The pharmaceutically acceptable vehicles used were: propylene glycol (PG), ethyl lactate (EL), or N-methylpyrrolidone (NMP).

In vitro skin permeation studies were conducted using modified Franz diffusion cells to determine the delivery of estrogens and progestins from various drug polymer matrices. The release liner was peeled off of the drug matrix layer. The drug matrix layer was then gently pressed onto the stratum corneum of heat separated epidermis from human cadaver skin. This skin membrane with the backing and drug matrix affixed thereto was then mounted between the two half-cells and fastened with a clamp. The receiver compartment was filled with 1% hydroxylpropylcyclodextrin in normal saline solution with 0.01% sodium azide. The temperature was maintained at 32° C. Samples were taken at preset intervals and assayed by HPLC. The flux was calculated from the slope of the cumulative amounts of the drug in the receiver compartment versus time.

| | | Lactic | | | Permeation | Duro-Tak ® |
|---|---|---|---|---|---|---|
| # | NGMN | Acid | Vehicle | | enhancer | 87-4287 |
| 1 | 2 | — | — | | — | 89 |
| 2 | 2 | 10 | 20 | (PG) | — | 68 |
| 3 | 2 | 10 | 20 | (PG) | 10 lauryl lactate | 58 |
| 4 | 2 | 10 | 20 | (EL) | 10 lauryl lactate | 58 |
| 5 | 2 | 10 | 20 | (NMP) | 10 lauryl lactate | 58 |

| | | | | | |
|---|---|---|---|---|---|
| 6 | 2 | 10 | — | 10 lauryl lactate | 78 |
| 7 | 2 | — | — | 20 lauryl lactate | 78 |

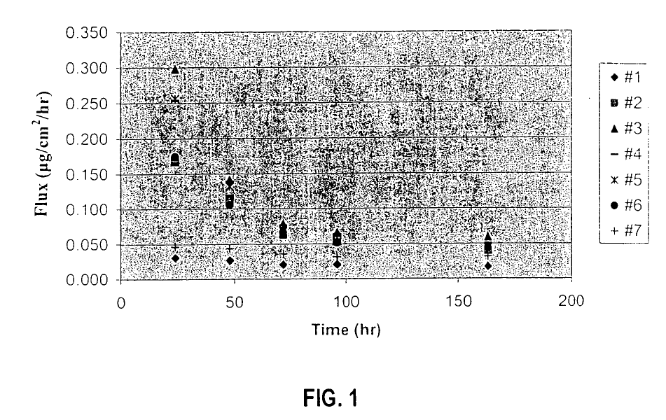

The results are depicted in FIG. 1, and show that the formulations of the invention, formulations #2-6, provided improved drug flux as compared to test formulations #1 and #7, which did not contain lactic acid. Test formulation #1 also did not contain a pharmaceutically acceptable vehicle or permeation enhancer.

### Example 2

The in vitro flux of norelgestromin (NGMN) and ethinyl estradiol (EE) from the following formulation was evaluated, using the in vitro skin permeation method set forth in Example 1. This formulation also included crosslinked polyvinyl pyrrolidone (PVP) as a matrix modifier.

| Formulation #43 | wt % |
|---|---|
| NGMN | 8 |
| EE | 1 |
| Lactic acid | 3 |
| Labrafil | 10 |
| PG | 10 |
| PVP, crosslinked | 10 |
| Duro-Tak ® 87-4287 | 58 |

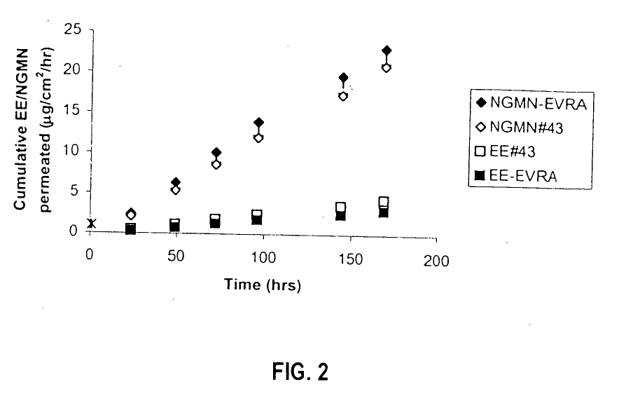

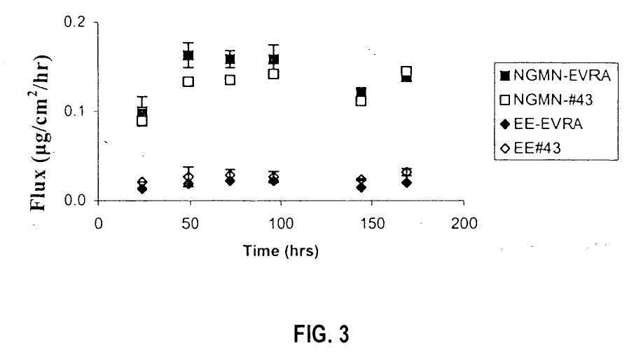

The in vitro flux of NGMN and EE flux from the Ortho Evra® norelgestromin/ethinyl estradiol transdermal system (Ortho-McNeil Pharmaceutical, Inc.) was also measured. The results are depicted in FIG. 2 and FIG. 3, and show that the formulation of the invention provided flux data comparable to that of the commercial formulation.

### Example 3

The in vitro flux of norelgestromin (NGMN) and ethinyl estradiol (EE) from the following formulation was evaluated, using the in vitro skin permeation method set forth in Example 1.

| Formulation #46 | wt % |
|---|---|
| NGMN | 8 |
| EE | 1 |
| Lactic acid | 5 |
| Lauryl lactate | 10 |
| PVP, crosslinked | 10 |
| Duro-Tak ® 87-4287 | 66 |

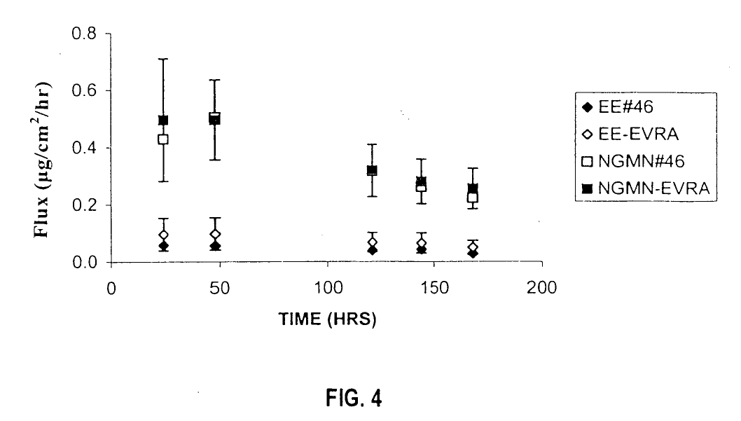

The in vitro flux of NGMN and EE flux from the Ortho Evra® transdermal system was also measured. The results are depicted in FIG. 4, and show that the formulation of the invention provided flux data comparable to that of the commercial formulation.

### Example 4

The in vitro flux of norelgestromin (NGMN) and ethinyl estradiol (EE) from the following formulation was evaluated, using the in vitro skin permeation method set forth in Example 1.

| Formulation #110E | wt % |
|---|---|
| NGMN | 10 |
| EE | 2 |
| Lactic acid | 2 |
| Lauryl lactate | 4 |
| PVP, crosslinked | 10 |
| Duro-Tak ® 87-900A | 72 |

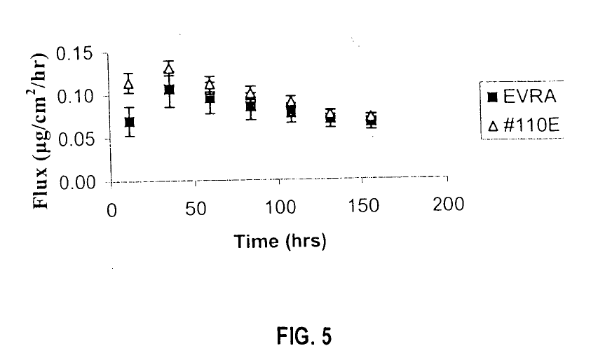

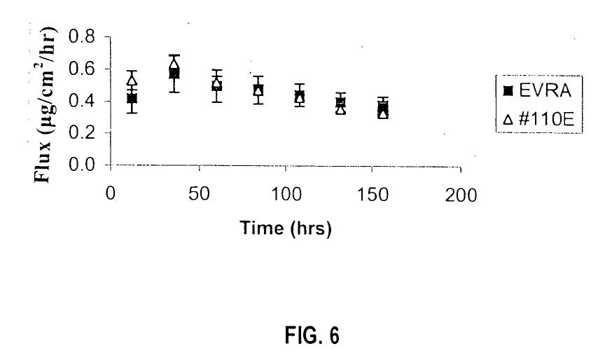

The adhesive, Duro-Tak® 87-900A, was provided with an ethyl acetate solvent. Loparex PET, a clear polyester liner with a silicone coating, was used as the release liner. 3M™ Scotchpak™ 9723, a material having a pigmented polyethylene layer and a layer of polyester, was used as the backing layer. FIG. 5 depicts the in vitro skin flux of EE and FIG. 6 depicts the in vitro skin flux of NGMN for Formulation # 110E, as compared to the Ortho Evra® transdermal system.

### Example 5

The in vitro flux of norelgestromin (NGMN) and ethinyl estradiol (EE) from the following formulations was evaluated, using the in vitro skin permeation method set forth in Example 1.

| | Formulation | Formulation |
|---|---|---|
| Component | #91E (wt %) | #107C (wt %) |
| NGMN | 10 | 8.5 |
| EE | 2 | 1.5 |
| Lactic acid | 2 | 2 |
| Labrafil | 4 | |
| Lauryl lactate | — | 4 |
| PVP, crosslinked | 10 | 8 |
| Duro-Tak ® 87-900A | 72 | 76 |

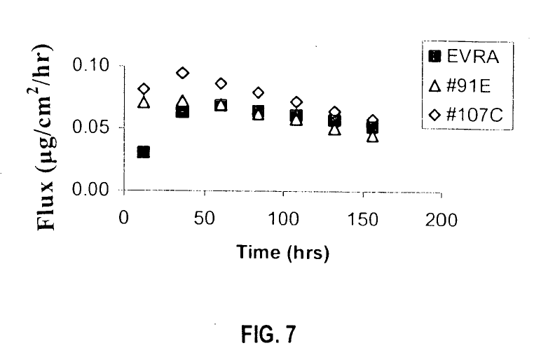

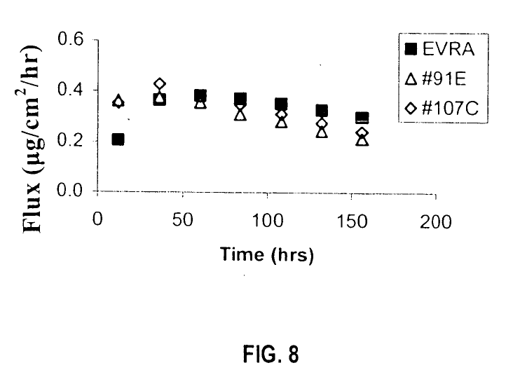

FIG. 7 depicts the in vitro skin flux of EE and FIG. 8 depicts the in vitro skin flux of NGMN for Formulation #91 E and #107C, as compared to the Ortho Evra® transdermal system.

### Example 6

The in vitro flux of norelgestromin (GMN) and ethinyl estradiol (EE) from the following formulations was evaluated, using the in vitro skin permeation method set forth in Example 1.

| | Formulation | Formulation |
|---|---|---|
| Component | 1 (wt %) | 2 (wt %) |
| NGMN | 11 | 11 |
| EE | 2 | 2 |
| Lactic acid | 2 | 2 |
| Lauryl lactate | 8 | 8 |
| PVP, crosslinked | 10 | 20 |
| Duro-Tak ® 87-900A | 67 | 57 |

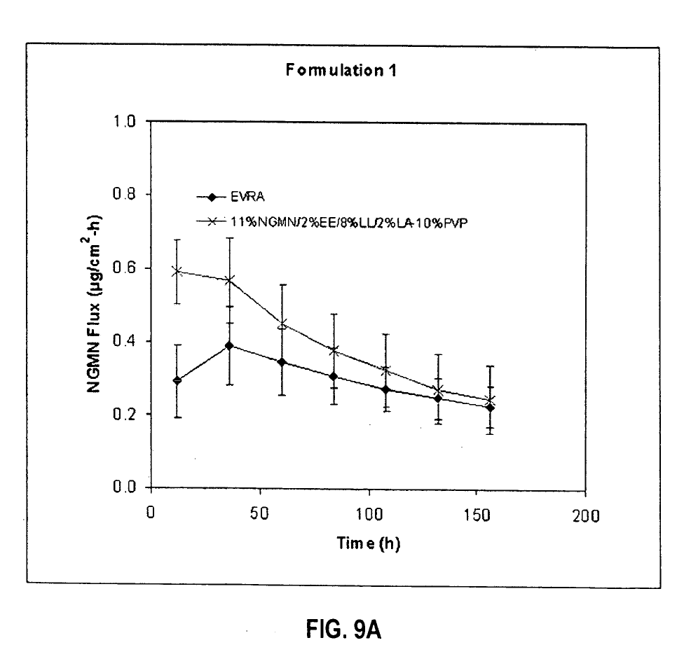

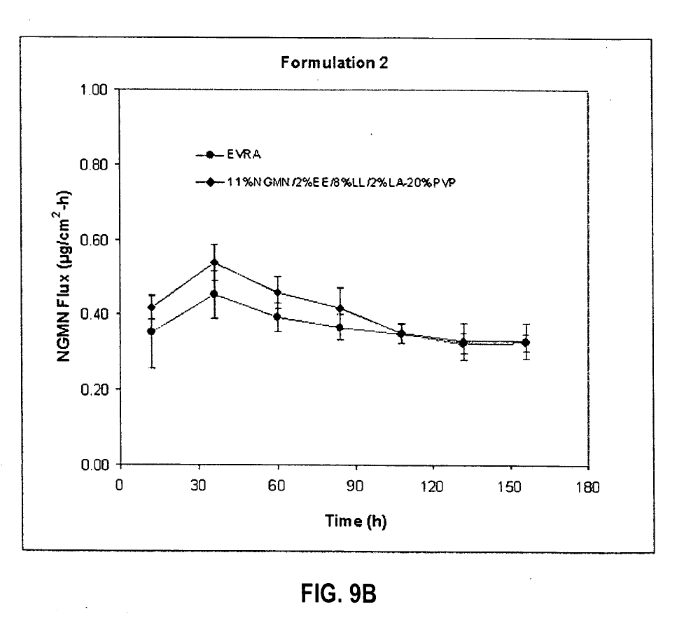

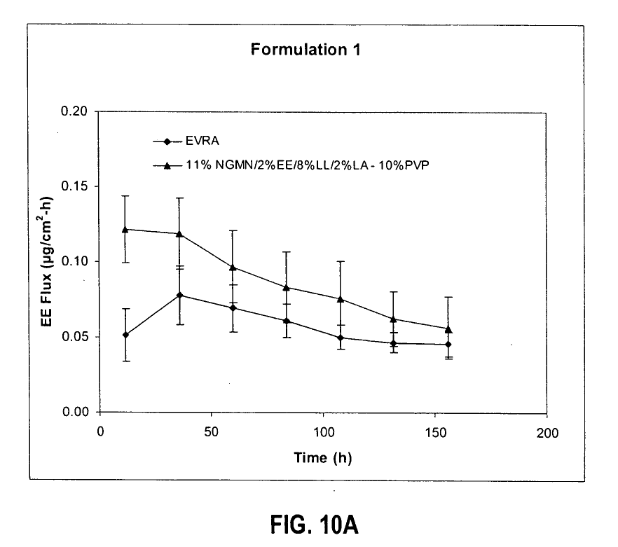

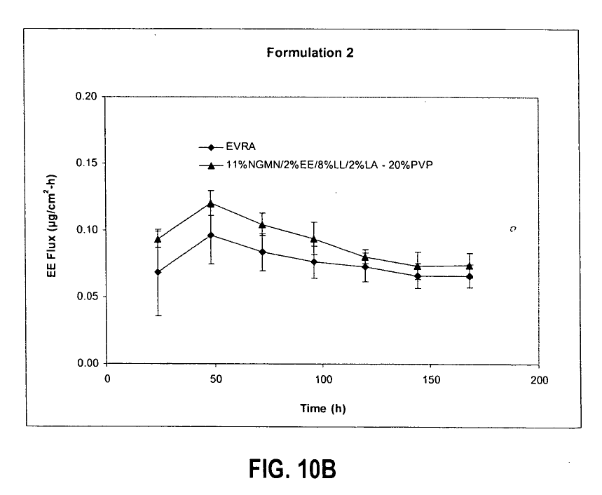

FIGS. 9A and 9B depict the in vitro skin flux of NGMN for formulations 1 and 2 of the table given above, as compared to the Ortho Evra® transdermal system. FIGS. 10A and 10B depict the in vitro skin flux of EE for formulations 1 and 2 of the table given above, as compared to the Ortho Evra® transdermal system.

All patents, patent applications, journal articles and other references cited herein are incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

It is to be understood that while the invention has been described in conjunction with the preferred specific embodiments hereof, the foregoing description, as well as the examples which are intended to illustrate and not limit the scope of the invention, it should be understood by those skilled in the art that various changes may be made and equivalents may be substituted without departing from the scope of the invention. Other aspects, advantages and modifications will be apparent to those skilled in the art to which the invention pertains.

Accordingly, the scope of the invention should therefore be determined with reference to the appended claims, along with the full range of equivalents to which those claims are entitled.

We claim:

1. A monolithic transdermal system for the administration of at least one active agent, comprising a drug reservoir laminated to a backing layer, wherein the drug reservoir comprises:
an effective amount of an active agent selected from estrogens, progestins, and combinations thereof;
a permeation enhancing amount of an organic acid having a molecular weight in the range of about 60 to about 200; and
a pharmaceutically acceptable vehicle,
in an adhesive matrix composed of a skin contact adhesive selected from acrylate adhesives, silicone adhesives, and polyisobutylene adhesives.

2. The transdermal system of claim 1, wherein the drug reservoir comprises a combination of an estrogen and a progestin.

3. The transdermal system of claim 2, wherein the estrogen is ethinyl estradiol and the progestin is norelgestromin.

4. The transdermal system of claim 2, wherein the drug reservoir further includes an androgen.

5. The transdermal system of claim 4, wherein the androgen is testosterone, a testosterone ester, DHEA, or 4-DHT.

6. The transdermal system of claim 1, wherein the organic acid is an alpha-hydroxy acid.

7. The transdermal system of claim 6, wherein the alpha-hydroxy acid is selected from lactic acid, glycolic acid, citric acid, tartaric acid, and malic acid.

8. The transdermal system of claim 7, wherein the alpha-hydroxy acid is lactic acid.

9. The transdermal system of claim 8, wherein the lactic acid represents about 0.5 wt. % to about 15 wt. % of the drug reservoir.

10. The transdermal system of claim 8, wherein the lactic acid represents about 2 wt. % to about 10 wt. % of the drug reservoir.

11. The transdermal system of claim 8, wherein the lactic acid represents about 1 wt. % to about 5 wt. % of the drug reservoir.

12. The transdermal system of claim 1, wherein the vehicle represents about 2 wt. % to about 40 wt. % of the drug reservoir.

13. The transdermal system of claim 1, wherein the vehicle represents about 2 wt. % to about 20 wt. % of the drug reservoir.

14. The transdermal system of claim 13, wherein the vehicle is selected from C₁₋₁₈ branched, linear, cyclic, saturated and unsaturated monohydric alcohols; polyols and esters thereof; N-methylpyrrolidone; and C₁₋₄ alkanol esters of lactic acid; and combinations thereof.

15. The transdermal system of claim 14, wherein the vehicle is a polyol.

16. The transdermal system of claim 15, wherein the polyol is propylene glycol.

17. The transdermal system of claim 13, wherein the vehicle is N-methylpyrrolidone.

18. The transdermal system of claim 13, wherein the vehicle is a C₁₋₄ alkanol ester of lactic acid.

19. The transdermal system of claim 18, wherein C₁₋₄ alkanol ester of lactic acid is ethyl lactate.

20. The transdermal system of claim 1, wherein the vehicle is an additional permeation enhancer selected from: alcohols; alkanones; alkanones; amides and other nitrogenous compounds; 1-substituted azacycloheptan-2-ones; bile salts; cholesterol; cyclodextrins and substituted cyclodextrins; ethers; saturated and unsaturated fatty acids; saturated and unsaturated fatty acid esters; saturated and unsaturated fatty alcohol esters; glycerides and monoglycerides; organic acids; methyl nicotinate; pentadecalactone; polyols and esters thereof; phospholipids; sulfoxides; surfactants; terpenes; and combinations thereof.

21. The transdermal system of claim 20, wherein the permeation enhancer is selected from saturated and unsaturated fatty alcohol esters, and glycerides.

22. The transdermal system of claim 21, wherein the permeation enhancer is selected from lauryl lactate, labrafil and triacetin.

23. The transdermal system of claim 1, wherein the active agent represents about 1 wt. % to 20 wt. % of the drug reservoir.

24. The transdermal system of claim 1, wherein the drug reservoir further includes an adhesive matrix modifier.

25. The transdermal system of claim 25, wherein the matrix modifier is cross-linked polyvinyl pyrrolidone.

26. A method for preventing ovulation in a mammalian female, comprising applying a transdermal drug delivery system to a body surface of the female for a predetermined time period, wherein the system comprises a drug reservoir laminated to a backing layer, the drug reservoir comprising effective ovulation-preventing amounts of an estrogen and a progestin, a permeation enhancing amount of a low molecular weight organic acid, and a pharmaceutically acceptable vehicle in an adhesive matrix composed of a skin contact adhesive selected from acrylate adhesives, silicone adhesives, and polyisobutylene adhesives.

27. The method of claim 26, wherein the estrogen is ethinyl estradiol and the progestin is norelgestromin.

28. The method of claim 27, wherein the ovulation-preventing amounts are effective to deliver about 10 μg/day to about 35 μg/day ethinyl estradiol and about 150 μg/day to about 350 μg/day norelgestromin.

29. A method for providing hormone replacement therapy to a mammalian female, comprising applying a transdermal drug delivery system to a body surface of the female for a predetermined time period, wherein the system comprises a drug reservoir laminated to a backing layer, the drug reservoir comprising effective hormone replacement amounts of an estrogen and a progestin, a permeation enhancing amount of a low molecular weight organic acid, and a pharmaceutically acceptable vehicle in an adhesive matrix composed of a skin contact adhesive selected from acrylate adhesives, silicone adhesives, and polyisobutylene adhesives.

30. The method of claim 26, wherein the estrogen is ethinyl estradiol and the progestin is norelgestromin.

31. The method of claim 27, wherein the effective hormone replacement amounts are effective to deliver about 10 μg/day to about 35 μg/day ethinyl estradiol and about 150 μg/day to about 350 μg/day norelgestromin.

32. A method for administering an estrogen, a progestin, or both, to a patient, comprising applying a transdermal drug delivery system to a body surface of the patient for a predetermined time period, wherein the system comprises a drug reservoir laminated to a backing layer, the drug reservoir comprising an estrogen, a progestin, or both an estrogen and a progestin, a permeation enhancing amount of a low molecular weight organic acid, and a pharmaceutically acceptable vehicle, in an adhesive matrix composed of a skin contact adhesive selected from acrylate adhesives, silicone adhesives, and polyisobutylene adhesives.

33. The method of claim 32, wherein the estrogen is ethinyl estradiol, the progestin is norelgestromin, and the organic acid is lactic acid.
